(19) United States
(12) Patent Application Publication
(10) Pub. No.: US 2007/0023541 A1
(43) Pub. Date: Feb. 1, 2007

(54) VOLATILE LIQUID DISSEMINATING APPARATUS

(75) Inventors: Colin Brown, Bracknell, Berkshire (GB); Guy Edward Naish, Bicester, Oxfordshire (GB); Anjum Fatima Shaukat, Twickenham, Middlesex (GB)

Correspondence Address: Andrew N. Parfomak;Norris McLaughlin & Marcus PA, 875 Third Avenue, 18th Floor, New York, NY, 10022, US

(73) Assignee: GIVAUDAN SA, Vernier (CH)

(21) Appl. No.: 11/457,518

(22) Filed: Jul. 14, 2006

(30) Foreign Application Priority Data
Jul. 18, 2005 (GB) 0514660.0

(51) Int. Cl. A61L 9/04

(52) U.S. Cl. 239/44; 239/34

(57) ABSTRACT

An apparatus adapted to disseminate a volatile liquid, such as a fragrance, an insecticide or a fungicide, into an atmosphere, the apparatus comprising a reservoir containing the liquid and a porous transfer member through which the liquid is conveyed from the reservoir to the atmosphere, the apparatus additionally comprising means of pressure equalization between atmosphere and reservoir, which means is adapted to effect a pressure-equalising change in volume of the reservoir, when the apparatus is in operation. The apparatus does not suffer from the leaking problems of known apparatus of the same kind, and is especially useful in apparatus in which one of two liquids, particularly fragrances, may be singly dispensed, by the inversion of the apparatus.

This invention relates to apparatus for disseminating volatile liquids into an atmosphere.

The presence in an atmosphere of a volatile liquid, such as a fragrance, an insecticide, a fungicide or a disinfectant, is often desired. A common way of achieving this is by means of a simple device comprising a reservoir containing the liquid and a porous liquid transfer member that serves both to remove the liquid from the reservoir and disseminate it into the atmosphere. The transfer member is typically a wick of any suitable material, such as porous plastics, ceramics and natural fibres. Such apparatus have found widespread acceptance.

In order for such an apparatus to function, it must have a means of pressure equalisation between reservoir and atmosphere, so that liquid dissemination can be continuous; without such means, the pressure in the reservoir would drop with loss of liquid and dissemination would stop long before the liquid is exhausted. Another aspect of pressure equalization is the reverse, excessive pressure inside the reservoir. This has the potential to occur when an apparatus is exposed to heat, for example, direct sunlight. Without a means of pressure equalization, the resultant increased pressure in the reservoir can force liquid into the transfer member at a rate that is higher than desirable and faster than it can be evaporated, meaning that it pools at the atmosphere end of the transfer member, a potential source of leakage. One typical way is to provide a small passage joining the atmosphere with the interior of the reservoir. In another method, the transfer member itself can be made sufficiently porous, so that the pressure can be equalised.

Such means are perfectly adequate for those apparatus that will remain in a single orientation for their entire service lives. However, this is not universally applicable. For example, there is a type of air freshener that is capable of disseminating two fragrances. This has usually the form of two conventional, independent, wick-type air fresheners mounted in 180° opposition to each other and joined at what would normally be considered their upper ends. Such two-fragrance air fresheners often resemble an hourglass, and like an hourglass they can be inverted and stood stably on a base at either end, the uppermost end becoming the fragrance disseminator via a dissemination surface connected to or forming part of the wick of the uppermost reservoir and positioned at or near the base. Dissemination from the wick of the uppermost reservoir only is ensured by partially encasing the wicks in both reservoirs in sleeves, such that the wick in the lower portion is shielded from liquid. A fragrance may therefore be changed by simply inverting the device.

It will readily be understood that the normal means of providing pressure equalization will not work in this case, as a conventional vent will allow the liquid to run out.

It has now been found that pressure equalization can be provided, and such a device can be made to work efficiently. The invention therefore provides an apparatus adapted to disseminate a volatile liquid into an atmosphere, the apparatus comprising a reservoir containing the liquid and a porous transfer member through which the liquid is conveyed from the reservoir to the atmosphere, the apparatus additionally comprising means of pressure equalization between atmosphere and reservoir, which means is adapted to effect a pressure-equalising change in volume of the reservoir, when the apparatus is in operation.

The invention further provides a method of disseminating a volatile liquid into an atmosphere, comprising the conveying of the liquid from a reservoir to the atmosphere by means of a porous liquid transfer member, the pressure between reservoir and atmosphere being equalised by changing the volume of the reservoir to an extent sufficient to equalize the pressure.

A feature of this invention is a reservoir whose volume is capable of being changed. The volume change does not necessarily occur in the reservoir itself, but may occur in an ancillary vessel which is connected to the reservoir and whose internal volume forms a single volume with that of the reservoir. For example, a flexible bladder may be attached to a relatively rigid reservoir by a tube. Such ancillaries are considered to be covered by the term “reservoir” for the purposes of this invention. However, for simplicity and cost effectiveness, it is preferred that the volume change take place in the reservoir itself.

This in turn means that at least part of the reservoir must be adapted to allow volume change. The skilled person will readily realise that there are many ways to achieve this, all of which fall within the scope of this invention. Some non-limiting examples include the following:
(a) a reservoir that is a bladder of a material that can be deformed,
(b) a reservoir of a rigid material, which incorporates flexible portions in its structure;
(c) a reservoir whose construction principles allow it to be varied in volume.

In the case of type (a), the reservoir may be somewhat like a bladder or balloon of a material that is deformable. Depending on the nature of the apparatus, this deformation may be elastic or non-elastic. Thus the material may be elastomeric (able to return to its original shape) when a force causing distortion is removed) or non-elastomeric (a material that can be stretched, but which remains permanently in the stretched state). In one embodiment, the bladder may be filled with liquid, and it progressively collapses as liquid is withdrawn, but which can also expand if the internal pressure increases above atmospheric pressure. This bladder may be housed in a suitable support structure that provides a base for standing, such as a rigid vessel or a cage.

In the case of type (b), examples include rigid vessels of any suitable material, such as glass, ceramic, plastics and metals, which have incorporated therein flexible portions. These flexible portions may be formed from any suitable material that is impervious to both gas and liquid. These can be, for example, flexible inserts that can be fitted into suitable openings in the reservoir. Such inserts may be made of any suitable material, such as rubber or plastics. When the reservoir is to be made of plastics material, the nature of the plastics material may allow the production of a rigid reservoir with flexible inserts in a single operation. For example, a plastics reservoir can be injection moulded such that the walls are generally thick enough to confer rigidity, but that these walls comprise thinner portions that are sufficiently flexible.

A particular variant of type (b) is basically type (a) reversed, that is, the liquid is contained in a rigid reservoir and the interior of the bladder is vented to the atmosphere, the reservoir, bladder and wick being arranged such that the liquid has no direct contact with the atmosphere. In this case, as liquid is withdrawn from the reservoir via the wick, the bladder expands into the reservoir to take up the pressure difference.

In the case of type (c), there are a number of possibilities. These include a folded or “concertina” structure, in which the reservoir can progressively expand with increasing external temperature or collapse as liquid is removed, the folds or concertina elements opening out or narrowing (possibly with concomitant elastomeric or non-elastomeric expansion or contraction of the material). Such a structure can be a bladder-type construction. Another possibility is provided, for example, by injection moulding a reservoir of plastics material. A further possibility is to have a reservoir with a telescoping structure. While somewhat more complex, some such structures have the advantages of being sufficiently rigid to be self-standing, and therefore to require no other support and of requiring the provision of no flexible inserts.

The nature of the liquid and the end-use will be decisive as to what kind of reservoir in desirable, and the skilled person can generally decide on an appropriate embodiment for any given case by simple experimentation. However, in the case of fragrances for air fresheners and the like, there are some important considerations to be borne in mind. Such materials are non-aqueous and very volatile, so the reservoir must be resistant to the contents, in the sense that it is not physically or chemically degraded by the contents. It must also contain the contents without appreciable loss. Many conventional materials otherwise suitable for reservoirs do not meet these conditions.

In such cases, the preferred materials are plastics laminates, that is, layers of plastics materials that have been laminated together to form a chemically-resistant and impermeable film. These are commonly found in the food packaging industry and typical examples include PA/PE, PA/EVOH/PE, PET/PE/EVOH/PE, PA/EVOH/EVA/PA and PET/Al/PE where PA=polyamide, PE=polyethylene, EVOH=ethylene-vinyl alcohol copolymer, PET=polyethylebe terephthalate, EVA=ethylene-vinyl acetate copolymer and Al=aluminium foil.

Such materials are flexible, but not elastomeric, so the reservoirs must be constructed such that they have the ability to reduce in volume. This may be done, for example, by providing the reservoirs with pleats, flutes or folds, such that a full reservoir has an outer surface substantially or completely without folds, but as liquid is disseminated, the surface folds and the reservoir loses volume.

A particular material for the purposes of this invention is a PA/EVOH/EVA/PA laminate of from 50-100 micrometres thickness. Although a range of thicknesses can be used, thickness for particular use with fragrances are generally from about 50 to about 100 micrometres, in particular about 60 micrometres. This gives adequate foldability and physical strength. A typical suitable commercially-available material is Amilen™ OX-80 ex Nabenhauer Verpackungen GmbH, Germany.

The liquid transfer member may be any suitable member. For example, it may be a porous wick of any suitable material, for example, compressed natural or artificial fibre, plastics, ceramics or metal. It may be moulded or stamped out of a sheet of material, or sintered from particulate materials

Although the invention may be used in connection with any such device, it is particularly useful in devices in which conventional vented construction is not possible, for example the “hour-glass”-type apparatus hereinabove described, in which the apparatus may be inverted. The invention therefore additionally provides an apparatus adapted to disseminate into an atmosphere one of two volatile liquids, the apparatus having, in use configuration, an upper and a lower reservoir, each containing one liquid and each equipped with a liquid transfer member, the apparatus being invertible and adapted to disseminate the liquid in the upper reservoir and prevent the dissemination of the liquid in the lower reservoir, the reservoirs being sealed from the atmosphere and each comprising a pressure-equalising structure that changes the volume of the reservoir such that the pressure between reservoir and atmosphere remains essentially the same.

In a particular embodiment of this apparatus, the liquids are fragrances and the reservoirs are bladders of impermeable laminated plastics materials whose structure comprises reservoir volume-reducing folds or flutes, as hereinabove described.

The structure of the apparatus is not critical, provided that the possibility of providing an upper and a lower reservoir is achievable. This may be achieved by any convenient means. Examples of such means include:
pivotally mounting the reservoirs on a base, such that the desired reservoir can be rotated into position;
having reservoirs that are sufficiently strong, rigid and stable to be self-supporting and providing a base at each end therein;
providing a rigid receptable, such as a container or a cage, within which a flexible reservoir may be mounted.

Such embodiments often overlap. For example, if a reservoir is non-rigid, for example, an elastomeric bladder, it might be advisable to enclose such a bladder in a rigid container, even if the reservoirs are pivotally mounted in a base. The skilled person will readily appreciate how various possibilities can be carried out.

It will be appreciated that, in such an apparatus, the liquid must be disseminated through what is the bottom of the reservoir, when the other liquid is being disseminated. This can be achieved by use of the ordinary skill of the art. For example, the bottom of the reservoir (when it is the lower reservoir) may comprise a dissemination surface that is attached to or forms part of the liquid transfer member. The dissemination of liquid when the reservoir is the upper reservoir and the prevention of leakage of liquid when this is the lower reservoir is achieved by any convenient means. One example of such means is by rendering liquid-tight the transfer member-reservoir interface and providing the liquid transfer member with a sleeve that is also in liquid-tight contact with the reservoir, the sleeve extending along the transfer member to an extent sufficient that, when the reservoir is the lower reservoir, the sleeve prevents contact of the liquid with the liquid transfer member.

Thus, when a reservoir is the lower reservoir, no liquid contacts the transfer member, and there is no liquid transfer. When the apparatus is inverted and the lower reservoir becomes the upper reservoir, the liquid again contacts that part of the transfer member that is not covered by the sleeve and liquid can again move up the transfer member for dissemination into the atmosphere.

Any potential for leakage at the time of a new inversion can be easily handled by conventional means, for example, by providing the lower reservoir adjacent to the liquid dissemination surface with a lid or a tray. This will cope with any residual liquid, which will soon evaporate and no more will be forthcoming, until re-inversion.

Apparatus according to this invention are easily manufactured using known materials and techniques, and they work very effectively. They may be made as single-use apparatus, or the reservoirs may be supplied as refills ready for insertion into an apparatus. They may also be made refillable by, for example, removing the transfer means, inverting the reservoir and adding liquid through a tube that extends into the reservoir.

The invention will now be further described with reference to the drawings, which depict preferred embodiments and which are not intended to be in any way limiting.

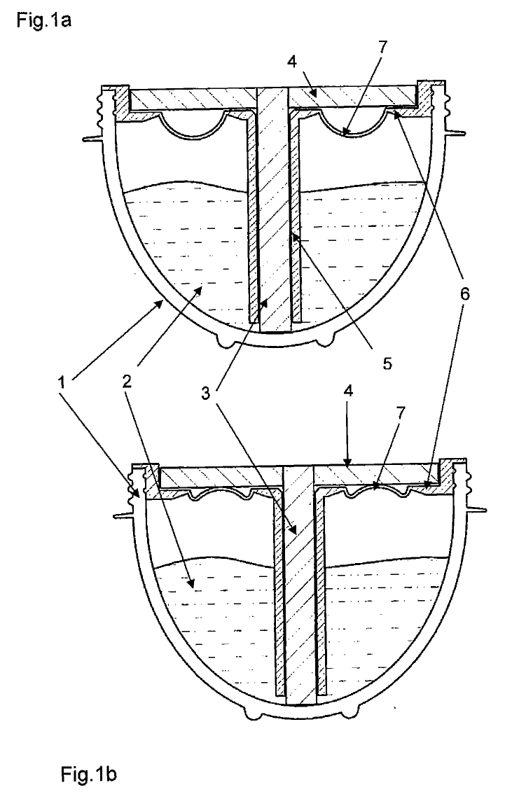

FIGS. 1a and 1b depict a schematic vertical cross section through one embodiment of an apparatus according to the invention, at two different stages. FIG. 1a depicts the embodiment in a ready-to-use state, and FIG. 1b depicts the same embodiment exposed to an elevated temperature.

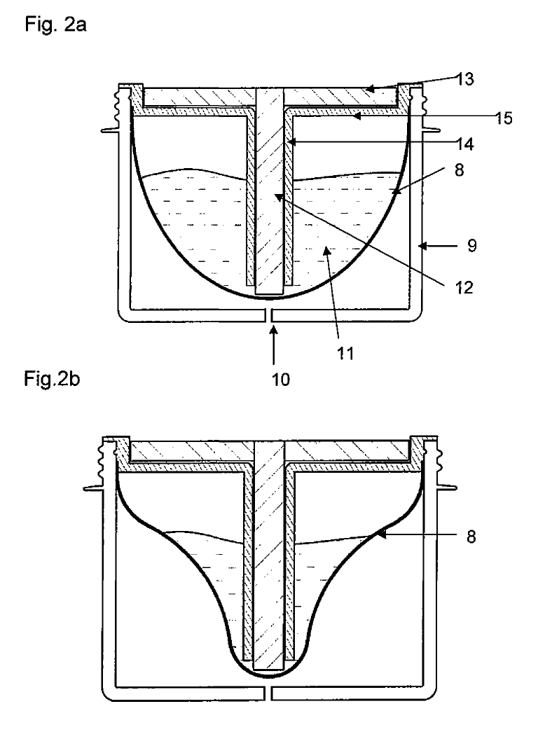

FIGS. 2a and 2b depict a schematic vertical cross-section through a further embodiment of the invention, depicting respectively a full reservoir and a partially empty reservoir.

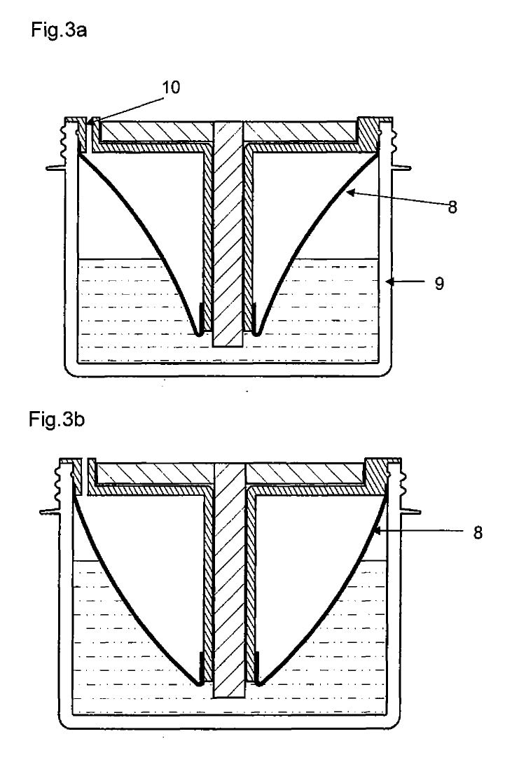

FIGS. 3a and 3b depict a schematic vertical cross-section through a further embodiment of the invention, depicting respectively a full reservoir and a partially empty reservoir.

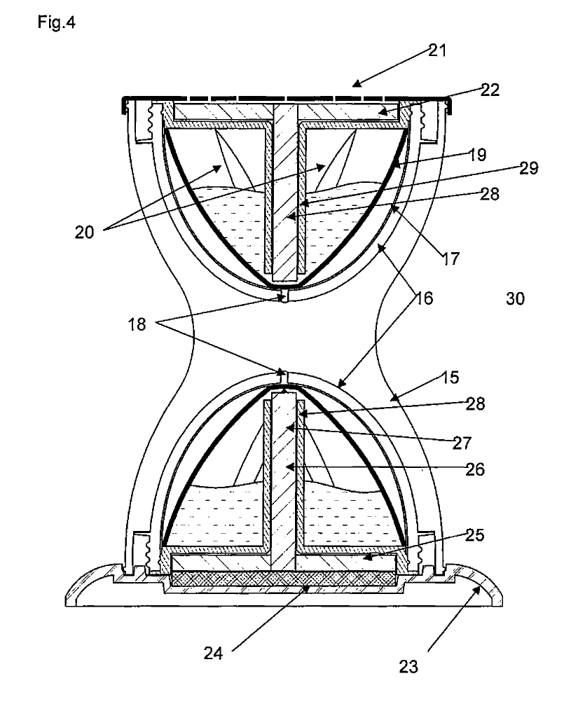

FIG. 4 depicts a double apparatus having an “hour glass” configuration.

In FIGS. 1a and 1b, a reservoir 1 of essentially circular transverse cross section contains a volatile liquid 2. This liquid is disseminated into the atmosphere by means of a wick 3, which is immersed at one end in the liquid. The wick terminates at its upper end in a planar dissemination member 4, made of the same material as the wick. The wick is held in place in the reservoir by an insert 5, which forms a sleeve around the wick for all of its length except the bottom portion, which is exposed to the liquid. Integral with the top of the sleeve is a radial extension 6, which extends outwardly from the sleeve to contact the reservoir in a liquid-tight fit, such that the liquid is sealed off from direct contact with the atmosphere. Formed within the insert on its radial extension is a circumferentially-arranged series of downwardly-pointing, essentially hemispherical protrusions 7. These are moulded into the insert, the material of the insert and the thickness of the protrusions being selected such that the protrusions are deformable.

Immediately prior to use, as shown in FIG. 1a, the protrusions are hemispherical. In the case of FIG. 1b, the apparatus has been exposed to elevated temperature, for example, by being exposed to direct sunlight. As a result, the internal pressure in the reservoir will rise. This rise in pressure is compensated for by the protrusions 6, which partially collapse under the pressure until the pressure in the reservoir is essentially equal to atmospheric pressure.

In FIG. 2a, there is depicted an apparatus in which the reservoir is a flexible bladder 8 of elastomeric material. This is suspended in a rigid container 9 having an air vent 10 at its base. The liquid 11 in the reservoir is withdrawn by a wick 12, which is equipped at its upper end with a planar member 13 of the same material. The wick is supported by an insert, which has a sleeve 14 portion that covers the wick for most of its length, leaving the bottom exposed to the liquid in the reservoir. At its upper end, the sleeve portion is contiguous with a radial closure portion that extends to the container, forming a liquid-tight seal and additionally securing the bladder.

In FIG. 2b, there is depicted the embodiment of FIG. 2a, in which some of the liquid has been disseminated. As a result, the pressure is equalised by the reduction in volume of the bladder and liquid continues to be disseminated.

FIGS. 3a and 3b show a variant on the bladder mechanism. In this case, the rigid container 9 is the liquid reservoir and the bladder 8 is vented to atmosphere through a vent 10 in the cap. The same mechanism is evident in FIG. 3b as is in FIG. 2b—in this case, as a response to the reduced pressure, the bladder expands into the rigid reservoir to equalise the pressure.

FIG. 4 depicts a double ended “hour-glass”-type device, used to disseminate two different volatile liquids, typically two different fragrances. This device basically comprises a body member 15 that has the form of two devices of the type depicted in FIG. 2a, joined in opposition to each other along a common longitudinal axis, each reservoir having the form of an essentially hemispherical dome 16 with an interior surface 17, and having a vent 18 for pressure equalisation. The device is designed to sit vertically with one reservoir vertically above the other, thus defining an upper reservoir and a lower reservoir, each with an associated bladder, wick, sleeve and evaporating surface.

In this embodiment, each bladder 19 is made of a non-elastomeric laminated plastics material and has a folded structure, somewhat like a coffee filter. The outwardly projecting folds 20 not only allow the bladder to expand and contract, but also to provide an air apace for pressure equalisation by holding the bladder 19 slightly apart from the respective interior surface 17 of the body member. The upper end of the device is equipped with a perforated cover plate 21, covering the upper evaporating surface 22 and the lower end with a base 23 capable of holding the device in an upright position. The base is equipped with an insert of absorbent material 24, which is adapted to oppose the lower evaporating surface 25 associated with the lower wick 26 and absorb any residual liquid. When the device is inverted, the cover plate 21 and the base 23 may simply be transferred to the new upper and lower ends.

In operation, the upper wick 28 is exposed to the liquid in the upper reservoir because the upper sleeve 29 does not extend to the bottom of the wick. Liquid can therefore transfer along the wick for dissemination into the atmosphere via the upper evaporating surface 22. At the lower reservoir, the liquid level is below the top of the lower sleeve 28, and thus no liquid can pass along the lower wick 26 to the lower evaporating surface 25 for dissemination into the atmosphere. When the device is inverted and the cover plate 21 and base 23 are exchanged, the lower and upper reservoirs and associated parts simply change places, and the liquid in what is now the upper reservoir is disseminated into the atmosphere.

The skilled person will readily appreciate that many variations of this invention can be made by simply applying the ordinary skill of the art, and that the previously described variants are by way of example only and are not meant to be limiting on the scope of the invention in any way.

1. An apparatus adapted to disseminate a volatile liquid into an atmosphere, the apparatus comprising a reservoir containing the liquid and a porous transfer member through which the liquid is conveyed from the reservoir to the atmosphere, the apparatus additionally comprising means of pressure equalization between atmosphere and reservoir, which means is adapted to effect a pressure-equalising change in volume of the reservoir, when the apparatus is in operation.

2. An apparatus according to claim 1, in which the means of pressure equalisation comprises a reservoir which is capable of volume contraction.

3. An apparatus according to claim 2, in which the material of the reservoir is elastomeric.

4. An apparatus according to claim 2, in which the reservoir is made of non-elastomeric material, and which is capable of volume reduction by means of flutes or folds incorporated into the reservoir, whereby the reservoir can fold, and therefore contract, as liquid is disseminated.

5. An apparatus according to claim 2, in which the material is a plastics laminate.

6. An apparatus according to claim 5, in which the laminate is a polyamide/ethylene-vinyl alcohol copolymer/ethylene-vinyl acetate copolymer/polyamide of thickness from about 50 micrometres to about 100 micrometres.

7. An apparatus according to claim 5, in which the thickness is about 60 micrometres.

8. An apparatus according to claim 1, in which the apparatus is an air freshener and the liquid is a fragrance.

9. An apparatus according to claim 1, in which the apparatus is adapted to disseminate one of two volatile liquids, the apparatus having, in use configuration, an upper and a lower reservoir, each containing one liquid and each equipped with a liquid transfer member, the apparatus being invertible and adapted to disseminate the liquid in the upper reservoir and prevent the dissemination of the liquid in the lower reservoir, the reservoirs being sealed from the atmosphere and each comprising a pressure-equalising structure that changes the volume of the reservoir such that the pressure between reservoir and atmosphere remains essentially the same.

10. An apparatus according to claim 9, in which the apparatus is an air freshener and the liquids are fragrances.

11. An apparatus according to claim 10, in which the reservoirs are made of non-elastomeric material, and which reservoirs are capable of volume reduction by means of flutes or folds incorporated into the reservoirs, whereby the reservoirs can fold, and therefore contract, as liquid is disseminated.

12. An apparatus according to claim 11, in which the material is a plastics laminate.

13. An apparatus according to claim 12, in which the laminate is a polyamide/ethylene-vinyl alcohol copolymer/ethylene-vinyl acetate copolymer/polyamide of thickness from about 50 micrometres to about 100 micrometres.

14. An apparatus according to claim 13, in which the thickness is about 60 micrometres.

15. A method of disseminating a volatile liquid into an atmosphere, comprising the conveying of the liquid from a reservoir to the atmosphere by means of a porous liquid transfer member, the pressure between reservoir and atmosphere being equalised by changing the volume of the reservoir to an extent sufficient to equalize the pressure.

16. A method according to claim 15, in which volume reduction is effected by means of a foldable reservoir, the reservoir comprising folds or flutes in its structure.
